(19) United States
(12) Patent Application Publication
(10) Pub. No.: US 2007/0129744 A1
(43) Pub. Date: Jun. 7, 2007

(54) Acupuncture needle guide assembly

(76) Inventors: Klaus Teichert, Pullach (DE); John Stan, Vancouver (CA)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/292,025

(22) Filed: Dec. 1, 2005

(51) Int. Cl. A61B 17/34

(52) U.S. Cl. 606/189

(57) ABSTRACT

An acupuncture needle guide assembly includes an acupuncture needle with a needle handle and a needle part, a guiding tube accommodating the acupuncture needle before use and providing guidance for setting the acupuncture needle, a holding tube being arranged in the guiding tube and providing a cover for the needle part of the acupuncture needle during setting thereof, and a detachable connector fixing the holding tube between the needle handle of the acupuncture needle and an inner wall of the guiding tube.

### BACKGROUND OF THE INVENTION

I. Field of the invention

The present invention relates generally to acupuncture devices and more particularly to an acupuncture needle guide assembly having a structure with a guiding tube accommodating an acupuncture needle and a holding tube covering the acupuncture needle. Furthermore, the present invention relates to an acupuncture method providing a precise placement of the needle and sterility of the needle before and during insertion into a body to be treated.

II. Description of the prior art.

Acupuncture treatment comprises a setting of an acupuncture needle into the skin of a patient at a predetermined sensitive region (acupuncture point). After setting, the acupuncture needle is further inserted through the skin into the body. Depending on the application and the region to be treated, the acupuncture needle can be inserted down to a depth of some cm. Accordingly, typical acupuncture needles have a length of about 5 cm to 10 cm or larger. As the needle part of the acupuncture needle has an extremely small diameter, a mechanical stabilization of the acupuncture needle is required during insertion.

U.S. Pat. No. 4,950,279 discloses an acupuncture delivery system comprising a needle which is arranged in a plastic dispensing tube. The dispensing tube provides a sterile protection of the needle before use as well as guidance for setting and inserting the needle into the skin. The application of the conventional acupuncture needle delivery system according to U.S. Pat. No. 4,950,279 is restricted to acupuncture needles with relatively short needles (about 6 cm and shorter). The dispensing tube can be used for setting the acupuncture needle only. For the further introduction of the needle into the body, this conventional device does not provide any mechanical stabilization.

An improvement in terms of further stabilization of the acupuncture needle even for large needles has been obtained with the needle assembly disclosed in U.S. Pat. No. 5,624,460. With this needle assembly, the acupuncture needle is fixed in a guide pipe with an adhesive membrane surrounding a needle handle at one hand of the guide pipe. At the opposite end of the guide pipe near the free tip of the acupuncture needle, a grip pipe has been arranged between a needle part of the acupuncture needle and the inner wall of the guide pipe. After setting the acupuncture needle and removing the guide pipe, the needle can be introduced into the body while manually gripping the grip pipe. The needle assembly of U.S. Pat. No. 5,624,460 has the following disadvantages. Firstly, the needle assembly has a complex structure which requires very precise dimensions of the guide and grip pipes. Furthermore, manufacturing of the needle assembly under sterile conditions is complex and expensive. Finally, the grip pipe represents a disturbance of the acupuncture treatment as the grip pipe increases the mass of the acupuncture needle.

### SUMMARY OF THE PRESENT INVENTION

The object of the invention is to provide an improved acupuncture needle guide assembly being capable to overcome the above-mentioned disadvantages and providing an acupuncture needle device having a simplified structure and maintaining the low weight of single acupuncture needles.

According to a first aspect, the present invention solves the above object by combining a guiding tube accommodating an acupuncture needle and a holding tube with a detachable connector being adapted for simultaneous fixing both a needle handle of the acupuncture needle and the holding tube. The connector is adapted for fixing the holding tube in a narrow space between the needle handle and an inner wall of the guiding tube. The provision of the connector represents an essential advantage in terms of improving the construction as well as the application of the needle guide assembly.

In an attached condition, all moveable parts of the acupuncture needle guide assembly of the invention are connected to each other with the detachable connector. Only one mechanical element is required for manufacturing the needle guide assembly.

Preferably, the connector is the only mechanical element fixing the moveable parts of the acupuncture needle guide assembly. If the connector is removed or broken, i.e., if the connector is a detached condition, all parts of the needle guide assembly are moveable relative to each other. Both the acupuncture needle and the holding tube can slide within the guiding tube for setting the acupuncture needle and further introducing it into a body to be treated.

According to a particularly preferred embodiment of the invention, the holding tube has an inner diameter being equal or greater than an outer diameter of the acupuncture needle handle. With this dimensioning, the holding tube can be removed from the acupuncture needle after insertion into the body so that a further deterioration of the acupuncture treatment is avoided. It is a surprising result of the present invention that the stability of setting and inserting the acupuncture needle is maintained even with the above dimensioning, i.e. with the holding tube having an inner diameter essentially greater than the needle diameter. The inventor has found that the holding tube with this dimensioning can be used for stable gripping or guiding the needle during insertion into the body.

For improving the stabilizing effect, the holding tube is preferably made of a flexible, bendable material. As an example, the holding tube is made of an elastic plastic or metal material. Alternatively, the holding tube can be made of a non-elastic material, like e.g., paper or foil.

According to a further preferred embodiment of the invention, the holding tube is made of a transparent material. In this case, advantages in terms of monitoring the progress of insertion can be obtained.

If the connector fixing the needle handle, the holding tube and the guiding tube together provides a clamp connection, there is an advantage of providing a very stable mechanical connection which can be obtained with a slight deformation of the above components only. Alternatively, the connector comprises an adhesive connection, which may have advantages for releasing the acupuncture needle within the guiding tube.

According to another aspect, the present invention overcomes the above-mentioned disadvantages by providing a method of acupuncture treatment using the acupuncture needle guide assembly according to the above first aspect. This method is particularly characterized by the step of releasing the acupuncture needle within the guiding tube by detaching the above connector. As the result of e.g., removing or breaking the connector, both the acupuncture needle and the holding tube are falling down towards the surface of the skin. The holding tube is automatically positioned such that one end thereof is in contact with the skin surface surrounding the acupuncture point to be treated. Preferably the holding tube is manually gripped after releasing the acupuncture needle within the guiding tube so that the acupuncture needle can be inserted into the body with sterile conditions.

### BRIEF DESCRIPTION OF THE DRAWINGS

The present invention will be more clearly understood by reference to the following detailed description when read in conjunction with the accompanying drawings in which:

FIG. 1 is a schematic sectional view of an acupuncture needle guide assembly according to the first embodiment of the invention; and

FIG. 2 is a perspective view of several phases of setting an acupuncture needle with a further embodiment of the acupuncture needle guide assembly of the invention.

### DETAILED DESCRIPTION OF THE PREFERRED EMBODIMENTS OF THE PRESENT INVENTION

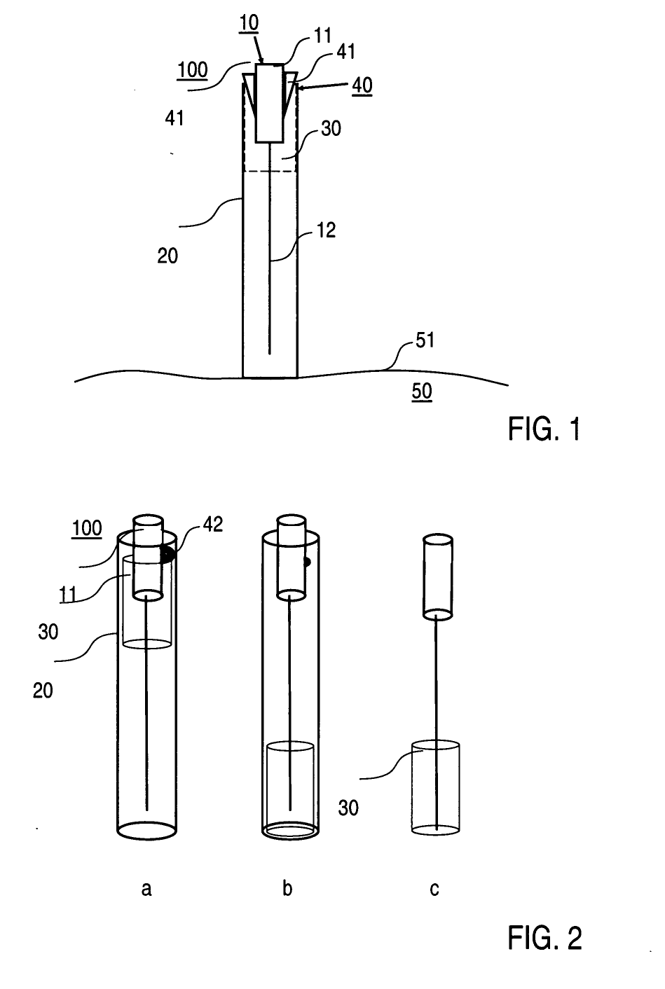

First, reference is made to FIG. 1 showing a cross sectional view of a needle guide assembly 100 comprising an acupuncture needle 10 with a needle handle 11 and a needle part 12, a guiding tube 20, a holding tube 30 and a connector 40. The assembly 100 is shown in a condition just before setting the acupuncture needle into the surface 51 of a human body 50 to be treated.

The acupuncture needle 10 comprises any needle structure as it is known in the art. As an example, the needle handle 11 is made of plastic having an axial length of 10 mm and a diameter of 2 to 3 mm. The needle part 12 is made of e.g., steel with an axial length of e.g., 8 cm and a diameter of 0.2 to 0.5 mm. The acupuncture needle 10 is arranged in the assembled condition such that the upper handle end of the needle handle 11 is protruding over the upper end of the guiding tube 20.

The guiding tube 20 comprises a plastic tube made of a transparent plastic, e.g., PE. The guiding tube 20 has an axial length of e.g., 10 cm, an outer diameter of e.g., 7 mm and an inner diameter of 5 mm.

The holding tube 30 is made of a thin, flexible material, e.g. plastic foil with a wall thickness of e.g., 100 μm to 0.5 mm. The outer diameter of the holding tube 30 is equal or slightly smaller than the inner diameter of the guiding tube 20, while the inner diameter of the holding tube 30 is equal or larger than the outer diameter of the needle handle 11. Accordingly, without the connector 40, all components 10, 20 and 30 would be slideable relative to each other.

The connector 40 comprises at least one, preferably three tapered keys 41 fixing the needle handle 11 in the center of the guiding tube 20 and clamping the holding tube 30 between the needle handle 11 and the inner wall of the guiding tube 20. The tapered keys 41 are made of e.g., plastic. They may be connected with each other with a plastic fiber for simultaneous removing the keys from the assembled condition. With one key, the needle handle and the holding tube would be eccentrically positioned.

FIGS. 2a, 2b and 2c illustrate several phases of setting an acupuncture needle with an acupuncture needle guide assembly 100 in a schematical perspective view. Like reference characters refer to like parts as in FIG. 1. In contrast to FIG. 1, the connector 40 is provided as an adhesive connection 42 connecting the needle handle 11, the guiding tube 20 and the holding tube 30.

As the first step of the method according to the invention, the assembly 100 is positioned on the skin of the body to be treated (not shown). This situation is illustrated in FIG. 2a. After the positioning step, the acupuncture needle is released by braking the adhesive connection 43 as shown in FIG. 2b in a situation where the needle handle 11 is gripped by the user while the holding tube 30 has been shifted down under the effect of gravity. After setting the acupuncture needle into the skin, the guiding tube is withdrawn as shown in FIG. 2c. The outer side of the holding tube 30 can be gripped manually while the acupuncture needle 10 is further introduced into the body.

Having described the present invention, many modifications thereto will become apparent to those skilled in the art to which it pertains without departing from the scope inspired of the present invention as defined in the appended claims.

What is claimed is:

1. An acupuncture needle guide assembly, comprising:
an acupuncture needle with a needle handle and a needle part;
a guiding tube adapted to accommodate the acupuncture needle before use and provide guidance for setting the acupuncture needle;
a holding tube being arranged in the guiding tube and providing a cover for the needle part of the acupuncture needle during setting thereof; and
a detachable connector fixing the holding tube between the needle handle of the acupuncture needle and an inner wall of the guiding tube.

2. The acupuncture needle guide assembly according to claim 1, wherein, with the connector in a detached condition, both the acupuncture needle and the holding tube are movable in the guiding tube.

3. The acupuncture needle guide assembly according to claim 1, wherein the holding tube has an inner diameter equal or greater than an outer diameter of the needle handle of the acupuncture needle.

4. The acupuncture needle guide assembly according to claim 1, wherein the holding tube is made of a flexible material.

5. The acupuncture needle guide assembly according to claim 1, wherein the holding tube is made of a transparent material.

6. The acupuncture needle guide assembly according to claim 1, wherein the connector comprises a clamp connection.

7. The acupuncture needle guide assembly according to claim 1, wherein the connector comprises an adhesive connection.

8. A method of introducing an acupuncture needle into a body to be treated, said method comprising the steps of:
providing the acupuncture needle guide assembly according to claim 1;
positioning the guiding tube with the acupuncture needle and the holding, tube on a surface of the body to be treated, wherein the needle part of the acupuncture needle is directed to the surface of the body;
releasing the acupuncture needle within the guiding tube by detaching a connector fixing the holding tube between the needle handle of the acupuncture needle and an inner wall of the guiding tube and shifting the acupuncture needle and the holding tube towards the surface of the body so that the holding tube is positioned with one end resting on the surface of the body;
setting the acupuncture needle so that the needle part is inserted into the surface of the body;
withdrawing the guiding tube; and
further introducing the acupuncture needle into the body, wherein the needle part is covered by the holding tube.

9. The method according to claim 8, wherein the step of further introducing the acupuncture needle comprises the step of manually gripping the holding tube.
